(19) United States
(12) Patent Application Publication
(10) Pub. No.: US 2019/0015594 A1
(43) Pub. Date: Jan. 17, 2019

(54) Drug Delivery Device

(71) Applicant: SANOFI, Paris (FR)

(72) Inventor: Anthony Paul Morris, Warwick, Warwickshire (GB)

(21) Appl. No.: 16/067,660

(22) PCT Filed: Jan. 4, 2017
(86) PCT No.: PCT/EP2017/050142

§ 371 Date: Jul. 2, 2018

(30) Foreign Application Priority Data
Jan. 4, 2016 (EP) 16305002.4

(51) Int. Cl. A61M 5/315; A61M 5/20

(52) U.S. Cl. CPC A61M 5/3155; A61M 5/31541; A61M 5/20

(57) ABSTRACT

It is provided a drug delivery device (1) comprising: a housing (4), a number sleeve (7) contained within the housing (4) and being rotatable with respect thereto and an axial retention mechanism (6) which is designed to axially restrain the number sleeve (7) to the housing (4) and to allow rotation of the number sleeve (7) with respect to the housing (4), wherein the axial retention mechanism (6) comprises a retention element (11). It is further provided a method of assembly of such a drug delivery device (1).

### TECHNICAL FIELD

The invention generally relates to a drug delivery device and to a method of assembly of such a drug delivery device.

### BACKGROUND

In certain types of drug delivery devices, such as pen-type devices, pre-filled cartridges are used. These cartridges are housed in a cartridge holder or housing. To dispense a certain set dose of a medicament contained in such cartridge, the drug delivery device has a dose setting element. During drug delivery, a piston rod coupled to the dose setting element presses against a piston (also commonly referred to as a “bung”, a “stopper”, or a “plunger”) contained within the cartridge in order to dispense the medicament through an attached needle assembly.

There remains a need for an improved drug delivery device respect to the related art.

### SUMMARY

An object of the present disclosure is to provide an improved drug delivery device and a method of assembly of such a drug delivery device.

The object is achieved by a drug delivery device according to claim 1 and by a method according to claim 15.

Exemplary embodiments are provided in the dependent claims.

In an exemplary embodiment, a drug delivery device comprises a housing, a number sleeve contained within the housing and being rotatable with respect thereto and an axial retention mechanism which comprises two bearings, in particular a retention element that axially restrains the number sleeve to the housing and allows rotation of the number sleeve with respect to the housing.

The retention mechanism improves the axial restraining of the number sleeve with respect to the housing by interlacing a number of radial abutments in order to prevent or impede an elastic deformation of the number sleeve, e.g. by influence of shock exposure, which may cause disengagement between the number sleeve and the housing. The drug delivery device may be configured as a dose-controlled injection device operable to deliver a variable, user-selectable dose of a medicament contained in the drug delivery device.

In an exemplary embodiment, the retention element comprises a hooked free end that may have an angled face angled about a transverse axis relative to a longitudinal axis, wherein the angled face forces the number sleeve to deform in a radial direction during assembly. The longitudinal axis runs in a direction of a longitudinal extension of the drug delivery device.

The retention mechanism may further comprise a retention radial abutment arranged on the housing and abutting against a circumference of the number sleeve. In particular, the retention abutment abuts against the circumference of the number sleeve that is located approximately opposite to a groove in the number sleeve. The groove may be engaged by the hooked free end.

In a further exemplary embodiment, a clearance between the hooked free end and the retention radial abutment is less than a wall thickness of the number sleeve. This leads to a lower risk of deformation of the number sleeve during storage, priming and/or operating the drug delivery device.

Furthermore, the retention radial abutment may be configured as a protrusion protruding radially outwards from a circumference, e.g. an outer circumference, of a housing retention element. The housing retention element may be coupled to the housing or may be configured as a separate part of the housing located within a distal portion of the number sleeve.

The retention mechanism may further comprise at least one restraining radial abutment with an angled face angled about a transverse axis relative to the longitudinal axis, wherein after the number sleeve is deformed in the radial direction by the hooked free end during assembly, the angled face forces the number sleeve to deform in an opposite radial direction back to an initial shape. A further deformation of the number sleeve during storage, priming and/or operating the drug delivery device is impeded.

Additionally, the retention mechanism may comprise at least one further restraining radial abutment arranged offset from the other restraining radial abutment in a circumferential direction. The further restraining element increases an engagement of the hooked free end and the grove and thus improves an efficiency of the retention mechanism.

Furthermore, the restraining radial abutment may be configured as a protrusion protruding radially outwards from a circumference of a housing restraining element. The housing restraining element may be coupled to the housing or may be configured as a part of the housing located within a distal portion of the number sleeve.

In an exemplary embodiment, the hooked free end may be coupled to the housing or may be configured as a part of the housing as well.

In an exemplary embodiment, the circumference of the number sleeve may comprise an inclined surface as an abutting surface for the restraining radial abutment in order to enable axial assembly.

In an exemplary embodiment, a method for assembly of a drug delivery device comprises the assembling of the number sleeve to the housing by displacing the number sleeve in a distal direction.

Further scope of applicability of the present invention will become apparent from the detailed description given hereinafter. However, it should be understood that the detailed description and specific examples, while indicating exemplary embodiments of the invention, are given by way of illustration only, since various changes and modifications within the spirit and scope of the invention will become apparent to those skilled in the art from this detailed description.

### BRIEF DESCRIPTION OF THE DRAWINGS

The present disclosure will become more fully understood from the detailed description given below and the accompanying drawings, which are given by way of illustration only, and do not limit the present disclosure, and wherein:

FIG. 1 is a schematic view of a simplified embodiment of a drug delivery device comprising a drug delivery mechanism and a retention mechanism,

FIG. 2 is an exploded view of a simplified embodiment of a drug delivery device,

FIG. 3 is a cut-out view of a part of an exemplary embodiment of the drug delivery device comprising the retention mechanism,

FIG. 4 is a magnified cut-out view of the part illustrated in FIG. 3,

FIG. 5 is a further cut-out view of a part of the drug delivery device,

FIG. 6 is a top view of a cross section of an exemplary embodiment of the drug delivery device,

FIG. 7 is a perspective view of a cross section of the exemplary embodiment of the drug delivery device of FIG. 6,

FIG. 8 is a cut out view of a part of an exemplary embodiment of the drug delivery device of FIG. 3 demonstrating deformation forces and

FIG. 9 is a top view of a cross section of an exemplary embodiment of the drug delivery device demonstrating a required deformation of the number sleeve.

Corresponding parts are marked with the same reference symbols in all figures.

### DETAILED DESCRIPTION

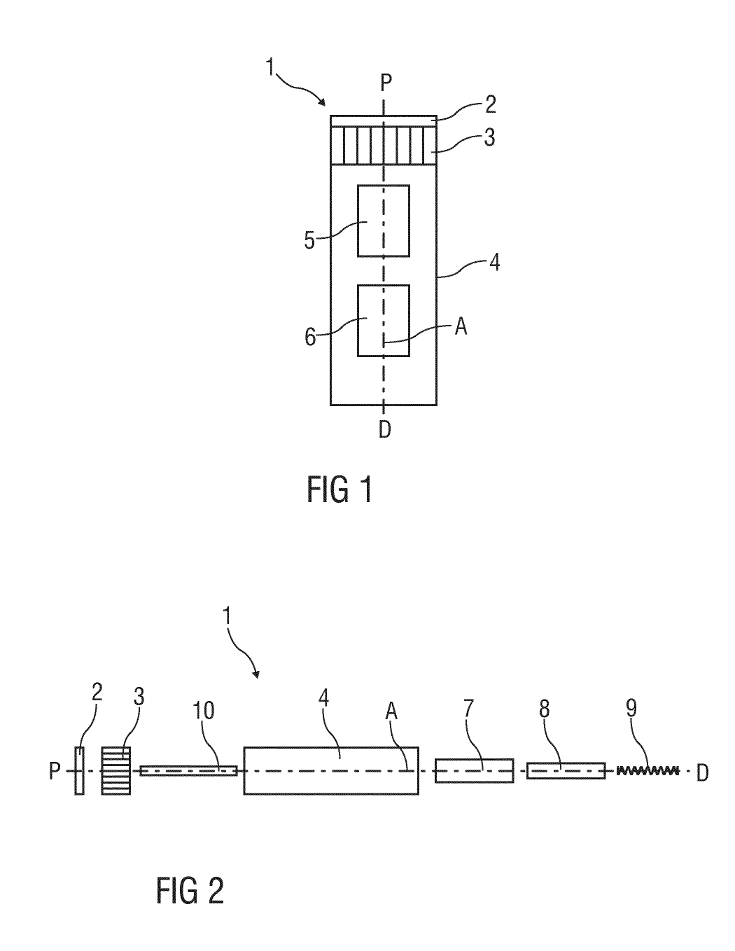

FIG. 1 schematically shows a simplified embodiment of a drug delivery device 1 that is configured as a pen-type device, in particular a dose-controlled injection device operable to deliver a variable, user-selectable dose of a medicament contained in the drug delivery device 1.

The drug delivery device 1 extends axially between a proximal direction P and a distal direction D. In the present application, the proximal direction P refers to a direction that under use of the drug delivery device 1 is located the furthest away from a drug delivery site of a patient. Correspondingly, the distal direction D refers to a direction that under use of the drug delivery device 1 is located closest to the drug delivery site of the patient.

According to the present embodiment, the drug delivery device 1 comprises a button 2, a dial grip 3, a housing 4, a drug delivery mechanism 5 and a retention mechanism 6. The drug delivery device 1 may comprise further components that are not shown in the present figures for reasons of clarity.

The button 2 forms a proximal end of the drug delivery device 1 and is permanently splined to the dial grip 3. Furthermore, the button 2 is releasably splined to a number sleeve 7 (illustrated in FIG. 2) that may be configured as a dose indicator comprising a window (not shown) that allows viewing a portion of the number sleeve 7 indicating an actual dose marking. The splined engagement between the button 2 and the number sleeve 7 is released, when the button 2 is pressed.

The dial grip 3 is a sleeve-like component with a serrated outer skirt and is axially constrained to the housing 4 and rotationally constrained to the button 2.

The housing 4 is a generally tubular element that provides receiving a liquid medicament, the drug delivery mechanism 5 and the retention mechanism 6.

FIG. 2 shows an exploded view of a simplified embodiment of the drug delivery device 1 comprising components of the drug delivery mechanism 5.

The drug delivery mechanism 5 is provided for delivery of the medicament stored in the housing 4 and comprises at least the number sleeve 7, a drive sleeve 8, a drive spring 9 and a piston rod 10. There may be more components provided than described below, but they are not further specified.

The number sleeve 7 may be configured as a tubular element that is marked with a sequence of numbers on a distal portion, which may be visible through a gauge element and openings (not shown) in the housing 4, to denote a selected dose of medicament. Thus, the number sleeve 7 may be configured as a dose setting member. The number sleeve 7 is rotated during dose setting and dose correction and during dose dispensing by the drive spring 9. The number sleeve 7 is furthermore axially constrained to the housing 4 by the retention mechanism 6 but allowed to rotate relative thereto.

The drive sleeve 8 may be a hollow member surrounding the piston rod 10 and arranged within the number sleeve 7. The drive sleeve 8 is axially movable relative to the housing 4, the piston rod 10 and the number sleeve 7 in the distal direction D against the bias of a not shown spring.

The drive spring 9 is attached at its distal end to the housing 4 and at the other end to the number sleeve 7. In particular, the drive spring 9 is located inside the number sleeve 7 and surrounds a distal portion of the drive sleeve 8.

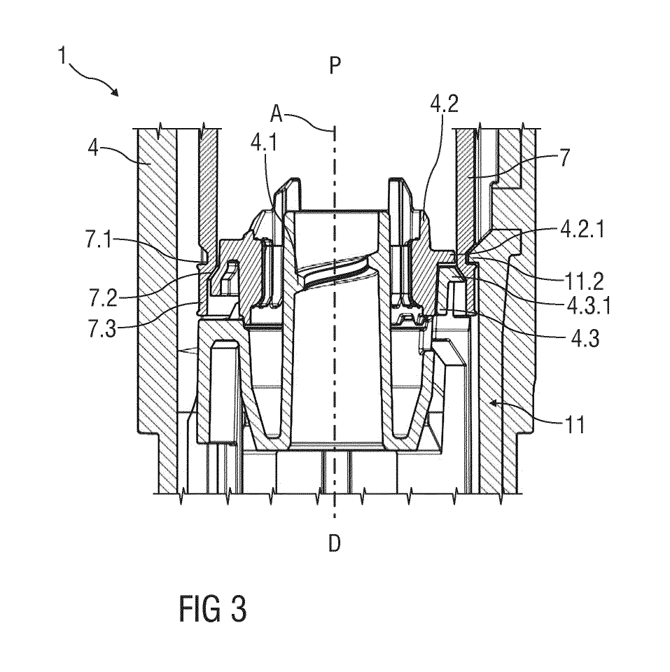

The piston rod 10 may be configured as an elongated member with a cylindrical shape and an inner thread that may engage a corresponding thread 4.1 of the housing 4 (illustrated in FIG. 3).

In order to activate the drug delivery mechanism 5, a user selects a variable dose of medicament by rotating the dial grip 3 clockwise, thereby generating an identical rotation of the number sleeve 7 with respect to the housing 4 and to the drive sleeve 8. A rotation of the number sleeve 7 causes the charging of the drive spring 9, thereby increasing energy stored within. The relative rotation of the drive sleeve 8 and the number sleeve 7 requires relatively low force or torque in a dose setting direction and requires a significantly higher force or torque in the opposite direction, particularly a dose correction direction.

As a dose is set, the user may activate the drug delivery mechanism 5 by depressing the button 2 in the distal direction D, thereby initiating dose dispensation.

As a result, the button 2 and the dial grip 3 are rotationally released from the number sleeve 7 and the drive spring 9. The drive sleeve 8 moves axially together with the button 2, thereby engaging the drive sleeve 8 to the number sleeve 7 such that relative rotation between the drive sleeve 8 and number sleeve 7 is prevented. Furthermore, the engagement between the housing 4 and the drive sleeve 8 releases, thus the drive sleeve 8 is allowed to rotate and is driven by the drive spring 9 via the number sleeve 7.

Rotation of the drive sleeve 8 causes rotation of the piston rod 10 which is axially translated due to its threaded engagement to the housing 4. Delivery of a dose of medicament continues while the user continues to depress the button 2.

Once the delivery of a dose is stopped and the number sleeve 7 indicates a zero dose, the user may release the button 2. When the user releases the button 2, the drive sleeve 8 is moved via a spring force to an initial or a rest position, thereby engaging the housing 4, preventing further rotation of the drive sleeve 8 relative to the housing 4 and stopping dose delivery. The drug delivery mechanism 5 may be provided with an audible and/or tactile feature at the end of dose delivery.

FIG. 3 schematically illustrates the retention mechanism 6, which is provided to axially constrain the number sleeve 7 to the housing 4 as mentioned before. Particularly, a cut-out view of the drug delivery device 1 in the area of the distal portion of the number sleeve 7 is illustrated.

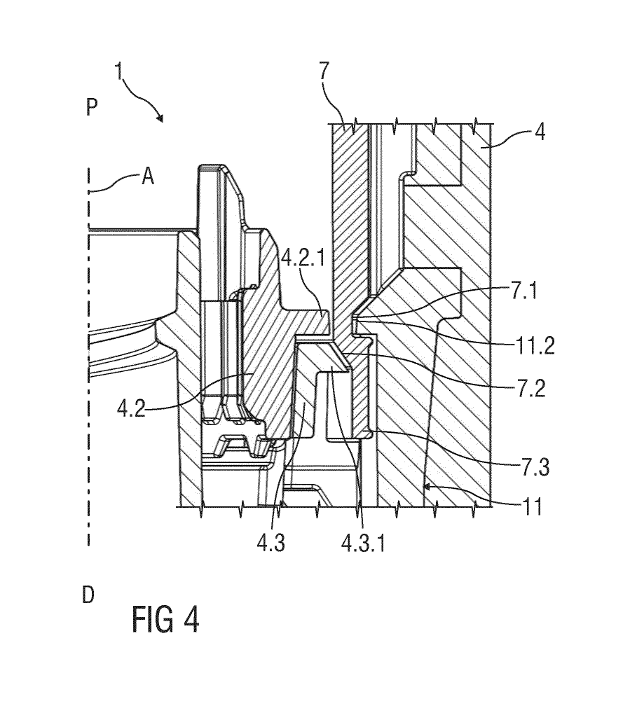

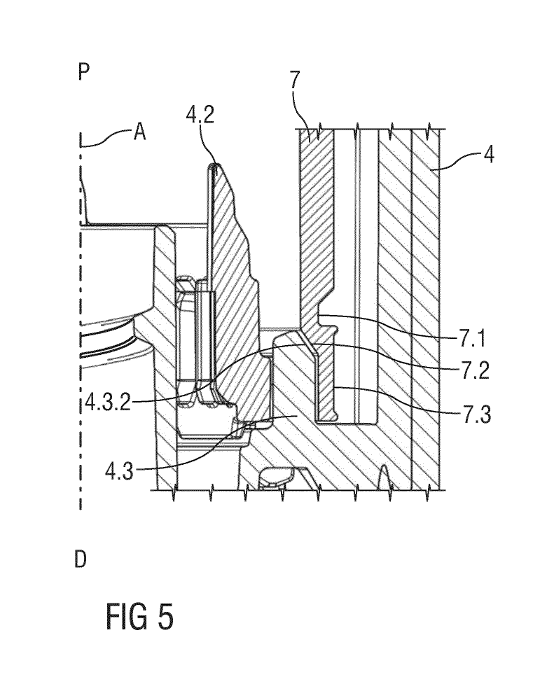

FIG. 4 schematically illustrates a magnified view of the longitudinal cut-out illustrated in FIG. 3.

Generally, the retention mechanism 6 comprises two axial bearings for retention after assembly. A first axial bearing is a distally facing surface on the hooked free end 11.2 abutting a proximally facing surface on the number sleeve 7. A second axially bearing is a proximally facing surface on the housing 4 abutting a distally facing surface on the number sleeve 7. The retention mechanism 6 will be described in more detail below.

The retention mechanism 6 comprises a circumferential groove 7.1 contained within the distal portion of the number sleeve 7 that engages a retention element 11 arranged between the housing 4 and the distal portion of the number sleeve 7. The retention mechanism 6 further comprises a housing retention element 4.2 and a housing restraining element 4.3.

The retention element 11 may be a separate component that is configured as a flexible arm comprising a hooked free end 11.2 that engages the groove 7.1 in the number sleeve 7. The retention element 11 may be inserted into a space between the housing 4 and the distal portion of the number sleeve 7. When the hooked free end 11.2 engages the groove 7.1, the retention element 11 latches with the number sleeve 7. For an easy assembly of the number sleeve 7, the hooked free end 11.2 is provided with a lead-in.

The housing retention element 4.2 may be coupled to or configured as a part of the housing 4 and arranged adjacent an inner circumference of the distal portion of the number sleeve 7. The housing retention element 4.2 is a ring-like component and provides a retention radial abutment 4.2.1 that abuts against a section of the inner circumference of the number sleeve 7 that is located opposite the groove 7.1 on the outer circumference of the number sleeve 7. The retention radial abutment 4.2.1 may be configured as a separate protrusion protruding radially outwards towards the inner circumference of the number sleeve 7.

The housing restraining element 4.3 may be configures as a separate component received within the housing 4 and fixed to the housing retention element 4.2, thereby providing a restraining radial abutment 4.3.1 abutting against an inclined surface 7.2 that is provided on the inner circumference of the number sleeve 7 located distally behind the groove 7.1. For assembly of the number sleeve 7, the restraining radial abutment 4.3.1 is provided with a lead-in.

The inclined surface 7.2 increases an inner diameter of the number sleeve 7 on a distal end 7.3 of the number sleeve 7, whereby the distal end 7.3 is configured as an axial protrusion of the number sleeve 7. Both, the retention radial abutment 4.2.1 and the restraining radial abutment 4.3.1 prevents a deformation of the number sleeve 7 in a radially inward direction, i.e. under shock exposure, thereby preventing a release of the hooked free end 11.2 out of the groove 7.1. Thus, the number sleeve 7 is axially constrained to the housing 4 in a reliable manner.

In order to increase the engagement of the hooked free end 11.2 in the groove 7.1, the housing restraining element 4.3 provides further restraining radial abutments 4.3.2 (illustrated in FIG. 5) arranged adjacent to the restraining radial abutment 4.3.1 respectively in a circumferential direction. Thus, the restraining radial abutment 4.3.1 is located between two further restraining radial abutments 4.3.2.

FIG. 5 illustrates a further longitudinal cut-out view of the drug delivery device 1, wherein the illustrated components are rotated about an angle of 30 degree around a longitudinal axis of the drug delivery device 1 with respect to the cut-out views illustrated in the FIGS. 3 and 4. Due to the rotational view, one of the further restraining radial abutments 4.3.2 can be seen.

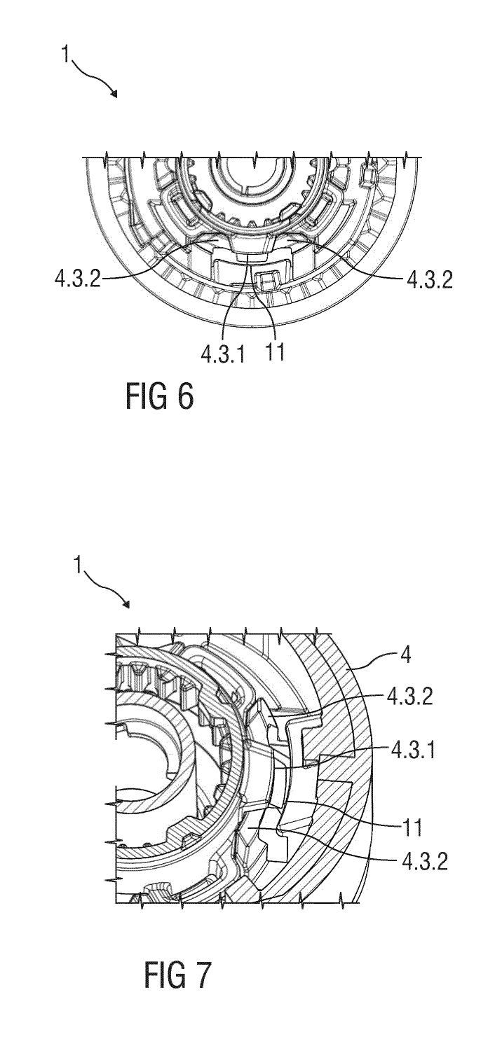

FIGS. 6 and 7 illustrate a cross section of the drug delivery device 1 respectively, wherein FIG. 6 is a top view of the cross section and FIG. 7 is a perspective view of the cross section.

The present figures illustrates the relative positions of the housing 4 and the housing restraining element 4.3 comprising the restraining radial abutment 4.3.1 and the further restraining radial abutments 4.3.2 and the retention element 11. The number sleeve 7 is not illustrated.

As a wall thickness of the number sleeve 7 is greater than a clearance between the hooked free end 11.2 and the retention radial abutment 4.2.1, both the housing retention element 4.2 and the housing restraining element 4.3 prevents a deformation of the number sleeve 7 that may release the hooked free end 11.2 out of the groove 7.1 in particular during a shock exposure.

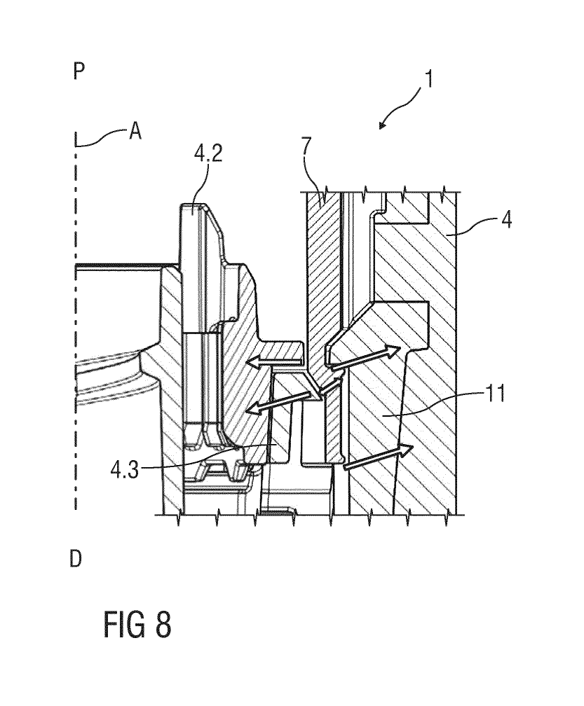

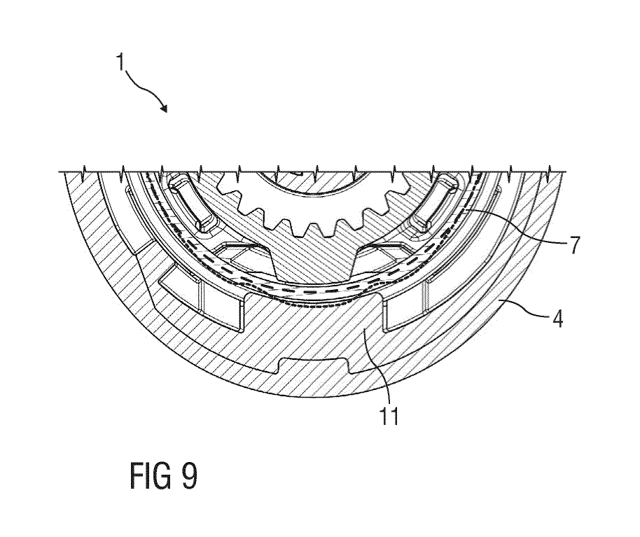

In order to disengage the retention element 11 and the number sleeve 7, a deformation of the housing retention element 4.2 and/or the housing restraining element 4.3 and the number sleeve 7 is required as illustrated in FIGS. 8 and 9.

FIG. 8 illustrates a longitudinal cut out view similar to FIG. 4. For reasons of clarity, not all reference signs are illustrated.

The required deformation forces for a disengagement of the retention element 11 and the number sleeve 7 are demonstrated with arrows. As can be seen, the components of the retention mechanism 6 are required to deform in a plurality of directions simultaneously in order to release the engagement between the number sleeve 7 and the retention element 11. Particularly, the retention radial abutment 4.2.1 is required to deform in a radially inward direction and/or the hooked free end 11.2 is required to deform in a radially outward direction, the distal end 7.3 of the number sleeve 7 is required to deform in a radially outward direction in order to separate the restraining radial abutment 4.3.1 from the inclined surface 7.2 and the distal portion of the number sleeve 7 is required to pass between the hooked free end 11.2 and the restraining radial abutment 4.3.1.

Thus, the number sleeve 7 is secured against axial movement with respect to the housing 4 even under shock exposure.

FIG. 9 illustrates a top view of a cross section of the drug delivery device 1.

The dotted line demonstrates a nominal form of the distal portion of the number sleeve 7. The dashed line demonstrates an approximated required form of the distal portion of the number sleeve 7 for disengagement between the number sleeve 7 and the retention element 11. The additional required deformations of the other components of the retention mechanism 6 are not shown.

The terms “drug” or “medicament” are used herein to describe one or more pharmaceutically active compounds. As described below, a drug or medicament can include at least one small or large molecule, or combinations thereof, in various types of formulations, for the treatment of one or more diseases. Exemplary pharmaceutically active compounds may include small molecules; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more of these drugs are also contemplated.

The term “drug delivery device” shall encompass any type of device or system configured to dispense a drug into a human or animal body. Without limitation, a drug delivery device may be an injection device (e.g., syringe, pen injector, auto injector, large-volume device, pump, perfusion system, or other device configured for intraocular, subcutaneous, intramuscular, or intravascular delivery), skin patch (e.g., osmotic, chemical, micro-needle), inhaler (e.g., nasal or pulmonary), implantable (e.g., coated stent, capsule), or feeding systems for the gastro-intestinal tract. The presently described drugs may be particularly useful with injection devices that include a needle, e.g., a small gauge needle.

The drug or medicament may be contained in a primary package or “drug container” adapted for use with a drug delivery device. The drug container may be, e.g., a cartridge, syringe, reservoir, or other vessel configured to provide a suitable chamber for storage (e.g., short- or long-term storage) of one or more pharmaceutically active compounds. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of a drug formulation (e.g., a drug and a diluent, or two different types of drugs) separately, one in each chamber. In such instances, the two chambers of the dual-chamber cartridge may be configured to allow mixing between the two or more components of the drug or medicament prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drug delivery devices and drugs described herein can be used for the treatment and/or prophylaxis of many different types of disorders. Exemplary disorders include, e.g., diabetes mellitus or complications associated with diabetes mellitus such as diabetic retinopathy, thromboembolism disorders such as deep vein or pulmonary thromboembolism. Further exemplary disorders are acute coronary syndrome (ACS), angina, myocardial infarction, cancer, macular degeneration, inflammation, hay fever, atherosclerosis and/or rheumatoid arthritis.

Exemplary drugs for the treatment and/or prophylaxis of diabetes mellitus or complications associated with diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the term “derivative” refers to any substance which is sufficiently structurally similar to the original substance so as to have substantially similar functionality or activity (e.g., therapeutic effectiveness).

Exemplary insulin analogues are Gly(A21), Arg(B31), Arg(B32) human insulin (insulin glargine); Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Exemplary insulin derivatives are, for example, B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin; B29-N-(N-lithocholyl-gamma-glutamyl)-des(B30) human insulin; B29-N-(w-carboxyheptadecanoyl)-des(B30) human insulin and B29-N-(w-carboxyheptadecanoyl) human insulin. Exemplary GLP-1, GLP-1 analogues and GLP-1 receptor agonists are, for example: Lixisenatide/AVE0010/ZP10/Lyxumia, Exenatide/Exendin-4/Byetta/Bydureon/ITCA 650/AC-2993 (a 39 amino acid peptide which is produced by the salivary glands of the Gila monster), Liraglutide/Victoza, Semaglutide, Taspoglutide, Syncria/Albiglutide, Dulaglutide, rExendin-4, CJC-1134-PC, PB-1023, TTP-054, Langlenatide/HM-11260C, CM-3, GLP-1 Eligen, ORMD-0901, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, TT-401, BHM-034. MOD-6030, CAM-2036, DA-15864, ARI-2651, ARI-2255, Exenatide-XTEN and Glucagon-Xten.

An exemplary oligonucleotide is, for example: mipomersen/Kynamro, a cholesterol-reducing antisense therapeutic for the treatment of familial hypercholesterolemia.

Exemplary DPP4 inhibitors are Vildagliptin, Sitagliptin, Denagliptin, Saxagliptin, Berberine.

Exemplary hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Exemplary polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20/Synvisc, a sodium hyaluronate.

The term “antibody”, as used herein, refers to an immunoglobulin molecule or an antigen-binding portion thereof. Examples of antigen-binding portions of immunoglobulin molecules include F(ab) and F(ab′)₂ fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region.

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, and multispecific antibodies (e.g., diabodies, triabodies, tetrabodies), minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Exemplary antibodies are anti PCSK-9 mAb (e.g., Alirocumab), anti IL-6 mAb (e.g., Sarilumab), and anti IL-4 mAb (e.g., Dupilumab).

The compounds described herein may be used in pharmaceutical formulations comprising (a) the compound(s) or pharmaceutically acceptable salts thereof, and (b) a pharmaceutically acceptable carrier. The compounds may also be used in pharmaceutical formulations that include one or more other active pharmaceutical ingredients or in pharmaceutical formulations in which the present compound or a pharmaceutically acceptable salt thereof is the only active ingredient. Accordingly, the pharmaceutical formulations of the present disclosure encompass any formulation made by admixing a compound described herein and a pharmaceutically acceptable carrier.

Pharmaceutically acceptable salts of any drug described herein are also contemplated for use in drug delivery devices. Pharmaceutically acceptable salts are for example acid addition salts and basic salts. Acid addition salts are e.g. HCl or HBr salts. Basic salts are e.g. salts having a cation selected from an alkali or alkaline earth metal, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are known to those of skill in the arts.

Pharmaceutically acceptable solvates are for example hydrates or alkanolates such as methanolates or ethanolates.

Those of skill in the art will understand that modifications (additions and/or removals) of various components of the substances, formulations, apparatuses, methods, systems and embodiments described herein may be made without departing from the full scope and spirit of the present invention, which encompass such modifications and any and all equivalents thereof.

### LIST OF REFERENCES

1 drug delivery device

2 button

3 dial grip

4 housing

4.1 thread

4.2 housing retention element

4.2.1 retention radial abutment

4.3 housing restraining element

4.3.1 restraining radial abutment

4.3.2 further restraining radial abutment

5 drug delivery mechanism

6 retention mechanism

7 number sleeve

7.1 groove

7.2 inclined surface

7.3 distal end

8 drive sleeve

9 drive spring

10 piston rod

11 retention element

11.2 hooked free end

A longitudinal axis

P proximal direction

D distal direction

1. A drug delivery device (1) comprising
a housing (4),
a number sleeve (7) contained within the housing (4) and being rotatable with respect thereto and
an axial retention mechanism (6), the axial retention mechanism comprising a retention element (11) and being configured for axially restraining the number sleeve (7) to the housing (4) and for allowing rotation of the number sleeve (7) with respect to the housing (4).

2. The drug delivery device (1) according to claim 1, wherein the retention element (11) axially restrains the number sleeve (7) to the housing (4) and allows rotation of the number sleeve (7) with respect to the housing (4).

3. The drug delivery device (1) according to any preceding claim, wherein the retention element (11) comprises a hooked free end (11.2).

4. The drug delivery device (1) according to any preceding claim, wherein
the axial retention mechanism (6) further comprises
a retention radial abutment (4.2.1) arranged on the housing (4) and abutting against a circumference of the number sleeve (7).

5. The drug delivery device (1) according to claim 4, wherein a clearance between the hooked free end (11.2) and the retention radial abutment (4.2.1) is less than a wall thickness of the number sleeve (7).

6. The drug delivery device (1) according to any one of the claims 3 to 5, wherein the hooked free end (11.2) engages a groove (7.1) in the number sleeve (7).

7. The drug delivery device (1) according to any one of the claims 4 to 6, wherein the retention radial abutment (4.2.1) is configured as a protrusion protruding radially outwards from an outer circumference of a housing retention element (4.2).

8. The drug delivery device (1) according to any one of the claims 3 to 7, wherein
the hooked free end (11.2) comprises an angled face angled about a transverse axis relative to a longitudinal axis (A) and
the angled face forces the number sleeve (7) to deform in a radial direction during assembly.

9. The drug delivery device (1) according to claim 8, wherein
the axial retention mechanism (6) further comprises at least one restraining radial abutment (4.3.1) with an angled face angled about a transverse axis relative to the longitudinal axis (A),
wherein after the number sleeve (7) is deformed in the radial direction by the hooked free end (11.2) during assembly, the angled face forces the number sleeve (7) to deform in an opposite radial direction back to an initial shape.

10. The drug delivery device (1) according to claim 9, wherein the axial retention mechanism (6) further comprises at least one further restraining radial abutment (4.3.2) arranged offset from the restraining radial abutment (4.3.1) in a circumferential direction.

11. The drug delivery device (1) according to any one of the claims 7 to 10, wherein the housing retention element (4.2) is coupled to the housing (4) or is configured as a separate part of the housing (4) located within a distal portion of the number sleeve (7).

12. The drug delivery device (1) according to any one of the preceding claims 9 to 11, wherein the restraining radial abutment (4.3.1) is configured as a protrusion protruding radially outwards from a circumference of a housing restraining element (4.3).

13. The drug delivery device (1) according to claim 12, wherein the housing restraining element (4.3) is coupled to the housing (4) or is configured as a part of the housing (4) located within a distal portion of the number sleeve (7).

14. The drug delivery device (1) according to any one of the preceding claims 3 to 13, wherein the hooked free end (11.2) is coupled to the housing (4) or is configured as a part of the housing (4).

15. The drug delivery device (1) according to any one of the preceding claims 9 to 14, wherein the circumference of the number sleeve (7) comprises an inclined surface (7.2) as an abutting surface for the restraining radial abutment (4.3.1).

16. Method of assembly of a drug delivery device (1) according to any one of the preceding claims, wherein the number sleeve (7) is assembled to the housing (4) by displacing the number sleeve (7) in a distal direction (D).
